SEQUENCE LISTING (22 sequences)

SEQ ID NO: 1 - DNA, 20 bp, Mus musculus
atggtgtccactagcatccc

SEQ ID NO: 2 - DNA, 23 bp, Mus musculus
cgatgttcaacttgcctgtgtag

SEQ ID NO: 3 - DNA, 20 bp, Mus musculus
atcgccatcgagagacaagt

SEQ ID NO: 4 - DNA, 20 bp, Mus musculus
agacaattccagcccaggat

SEQ ID NO: 5 - DNA, 20 bp, Mus musculus
gcctagcgggagagaaacct

SEQ ID NO: 6 - DNA, 19 bp, Mus musculus
ccgactgcgggaagagtgt

SEQ ID NO: 7 - DNA, 21 bp, Mus musculus
gccctcatcctagtggctatc

SEQ ID NO: 8 - DNA, 22 bp, Mus musculus
gcccagacattagaaccaaaga

SEQ ID NO: 9 - DNA, 21 bp, Mus musculus
tgtgcgctctatgcaaggatt

SEQ ID NO: 10 - DNA, 21 bp, Mus musculus
cacgctaagggtacgaagcag

SEQ ID NO: 11 - DNA, 23 bp, Mus musculus
tggcattaagaaggcccatatag

SEQ ID NO: 12 - DNA, 20 bp, Mus musculus
tgagcccctgacaggatgtc

SEQ ID NO: 13 - DNA, 22 bp, Mus musculus
ggtggaggctgacttcctgtat

SEQ ID NO: 14 - DNA, 20 bp, Mus musculus
cgacccgtcctttgaatttc

SEQ ID NO: 15 - DNA, 22 bp, Mus musculus
tgttcccctgaagagttcacaa

SEQ ID NO: 16 - DNA, 19 bp, Mus musculus
tttcgtgtacggccattgg

SEQ ID NO: 17 - DNA, 22 bp, Mus musculus
gggccaccacgctcttctgtct

SEQ ID NO: 18 - DNA, 22 bp, Mus musculus
gccactccagctgctcctccac

SEQ ID NO: 19 - DNA, 21 bp, Mus musculus
tggccaccttgttcagctacg

SEQ ID NO: 20 - DNA, 22 bp, Mus musculus
gccaaggccaaacacagcatac

SEQ ID NO: 21 - DNA, 26 bp, Mus musculus
caactacatggtctacatgttccagt

SEQ ID NO: 22 - DNA, 17 bp, Mus musculus
tgacccgtttggctcca

(19) United States
(12) Patent Application Publication
(10) Pub. No.: US 2010/0068200 A1
(43) Pub. Date: Mar. 18, 2010

(54) Methods and Compositions for Inhibiting Atherosclerosis and Vascular Inflammation

(75) Inventors: Timothy Tun Hla, Avon, CT (US); Athanasia Skoura, West Hartford, CT (US); Jason E. Michaud, Farmington, CT (US)

Correspondence Address: CANTOR COLBURN, LLP, 20 Church Street, 22nd Floor, Hartford, CT, 06103, US

(73) Assignee: THE UNIVERSITY OF CONNECTICUT, Farmington, CT (US)

(21) Appl. No.: 12/558,797

(22) Filed: Sep. 14, 2009

Related U.S. Application Data
(60) Provisional application No. 61/096,327, filed on Sep. 12, 2008.

(51) Int. Cl. A61K 39/395; A61K 48/00; A61K 31/437; A61P 9/10; A61P 29/00

(52) U.S. Cl. 424/130.1; 514/44 A; 514/303; 514/300

(57) ABSTRACT

Disclosed herein are compositions and methods for reducing inflammation associated with atherosclerosis and/or vascular inflammatory disease. The methods include administering to a subject in need of treatment for atherosclerosis and/or vascular inflammation a pharmaceutically effective amount of an inhibitor of the receptor activity of the S1P2 receptor. Also included are compositions including an S1P2 receptor antagonist and a pharmaceutically acceptable excipient.

### CROSS REFERENCE TO RELATED APPLICATIONS

This application claims priority from Provisional Application Ser. No. 61/096,327, filed Sep. 12, 2008, which is incorporated herein by reference in its entirety.

### STATEMENT REGARDING FEDERALLY SPONSORED RESEARCH & DEVELOPMENT

This invention was made with support from the United States Government under Grant # R37-HL67330 from the National Institutes of Health. The U.S. Government has certain rights in the invention.

### BACKGROUND

Risk factors for atherosclerosis promote endothelial injury, leading to lipoprotein deposition in the vessel wall. Sphingomyelin, a major constituent of lipoproteins, is metabolized by the sphingomyelinase pathway to produce sphingolipid metabolites, such as ceramide, sphingosine and sphingosine-1-phosphate (SIP) whose functional roles in vascular disease are not well understood. However, suppression of sphingolipid synthesis attenuates atherosclerosis in animal models and sphingolipid metabolites are considered a risk factor in human coronary artery disease. S1P is recently recognized as a multifunctional lipid mediator that signals via the SIP family of G protein-coupled receptors (S1P₁₋₅) and regulates vascular permeability, angiogenesis and immune cell trafficking.

What are needed are additional compositions and methods suitable for the treatment of atherosclerosis and other vascular inflammatory diseases.

### SUMMARY

In one embodiment, method of reducing inflammation associated with atherosclerosis and/or associated with a vascular inflammatory disease in a subject in need thereof, comprises administering to the subject in need of a reduction in inflammation associated with atherosclerosis and/or associated with a vascular inflammatory disease a pharmaceutically effective amount of an inhibitor of the activity of the S1P2 receptor or caspase-11.

In another embodiment, treating atherosclerosis includes inhibiting or reducing risk of cardiovascular and cerebrovascular diseases resulting from atherosclerosis, such as cardiac and/or cerebral ischemia, myocardial infarction, angina, peripheral vascular disease and stroke.

### BRIEF DESCRIPTION OF THE DRAWINGS

FIG. 1: En face analysis indicates reduction of atheroma in Apoe^{-/-}s1p2^{-/-} aortas (n=6) compared to Apoe^{-/-}s1p2^{+/+} (n=9, P<0.0001).

FIG. 2: Aortic root cross-sections of Apoe^{-/-}s1p2^{+/+} (n=5) and Apoe^{-/-} s1p2^{-/-} (n=5) stained with Hematoxylin Phloxin and Saffron (HPS) to characterize fibrotic areas, Trichrome to visualize collagen and Oil Red O to stain for lipid deposition. Lack of the receptor markedly reduced the plaque area (P<0.001).

FIG. 3: Aortic root sections from Apoe^{-/-}s1p2^{+/+} (n=3) and Apoe^{-/-}s1p2^{-/-} (n=3) were immunostained with MOMA-2. Macrophage infiltration was markedly diminished in the intima of Apoe^{-/-}s1p2^{+/+} mice (P<0.05).

FIG. 4: Loss of S1P₂ receptor in macrophages was sufficient to markedly reduce atherosclerosis (P<0.0001).

FIG. 5: MOMA-2 (macrophages) and Oil Red O (lipid accumulation) staining in lesions from Apoe^{-/-}s1p2^{+/+} (n=3) or Apoe^{-/-}s1p2^{-/-} (n=3) to Apoe^{-/-}s1p2^{+/+} after being on high cholesterol diet for 13 weeks exhibits that (d) lipid accumulation (P<0.04) and inflammatory component (P<0.03) of the plaque were significantly reduced in Apoe^{-/-}s1p2^{+/+} animals transplanted with Apoe^{-/-}s1p2^{-/-} bone marrow cells.

FIG. 6: S1p2r^{-/-} macrophages exhibit lower mRNA expression of caspase-11 compared to littermate controls under basal conditions (n=3, P<0.004). Upon LPS (1 μg/ml) stimulation for 6 h, caspase-11 gene expression is still significantly lower in S1p2r^{-/-} macrophages than in S1p2r^{+/+} treated cells (n=3, P<0.02).

FIG. 7: In contrast, caspase-1 expression is not significantly different between S1p2r^{-/-} and S1p2r^{+/+} treated BMDM.

FIG. 8: Pretreatment of elicited peritoneal macrophages with S1P₂ receptor specific antagonist JTE-013 (500 nM) blocks LPS (1 μg/ml) induced caspase-11 expression. Treatment of elicited peritoneal macrophages with oxLDL (50 μg/ml, 8 hrs) and TNFα (50 ng/ml, 8 hrs) upregulates caspase-11. oxLDL (50 μg/ml, 8 hrs)-dependent caspase-11 protein level increase is partially diminished by S1 P₂ antagonist JTE-013 (500 nM).

FIGS. 9, 10: Upon LPS (1 μg/ml) treatment, caspase-11 is detected in the complex immunoprecipitated by caspase-1 antibody in S1p2r^{+/+} BMDM. Caspase-1 co-immunoprecipitates with caspase-11 in LPS treated S1p2r^{+/+} BMDMs cells. In S1p2r^{-/-} cells, lack of expression of caspase-11 is reflected in reduced caspase-1-associated complex.

FIG. 11: Mice were treated with LPS (40 mg/kg) for 3 hrs and plasma cytokine levels were quantified (n=6-7). Mice pretreated with JTE-013 antagonist (1.2 mg/kg, 30 min pretreatment, n=7-9) had reduced serum IL-1β (P<0.003) and IL-18 (P<0.007) levels compared to mice treated with LPS. TNF-a levels were not changed significantly.

FIG. 12, 13: Upon LPS (40 mg/kg) injection, caspase-11 and IL-1β is detected in the complex immunoprecipitated by caspase-1 antibody in S1p2r^{+/+} spleen extracts. Caspase-11 and IL-1β were not part of caspase-1 inflammasome in S1p2r^{-/-} mice.

FIG. 14: Mice were treated with LPS (40 mg/kg) for 3 hrs and plasma cytokine levels were quantified. S1p2r^{-/-} mice had reduced serum IL-1β and IL-18 compared to control group S1p2r^{+/+} mice (n=6, P<0.001 and P<0.01, respectively). At 5 hrs of LPS treatment, the difference in serum IL-1β levels between WT and KO animals is still significant (n=6, P<0.02).

FIG. 15: Apoe^{-/-}S1p2r^{+/+} aortas from mice fed with high fat diet for 13 weeks present significantly higher expression of caspase-1 and caspase-11 compared to littermates controls that have been fed with regular chow diet. VCAM is also highly induced in aortas from high fat diet fed animals whereas eNOS expression is unaltered (n=4, *non-specific band detected in both groups of animals).

FIG. 16: Western blot analysis for caspase-11 (*non-specific band detected in both WT and KO aortae), caspase-1, IκBα and VCAM in Apoe^{-/-}S1p2r^{+/+} and Apoe^{-/-}S1p2r^{-/-} aortae from mice fed with “Western Diet” for 13 weeks. Caspase-1 and caspase-11 expression is reduced in Apoe^{-/-}S1p2r^{-/-} aortae whereas higher levels of IκBα were detected.

FIG. 17: En face Oil Red O staining analysis of aortae upon transplantation of S1p2r^{+/+} (n=5) or S1p2r^{-/-} (n=5) bone marrow cells to LDLr^{-/-}. Loss of S1P₂ receptor in bone marrow-derived cells was sufficient to markedly reduce atherosclerosis (P<0.05). Similarly, transplantation of casp11^{-/-} (n=1.3) bone marrow to LDLr^{-/-} animals significantly reduces Oil Red O positive atheromatic lesions compared to casp11^{+/+} (n=8) transplants (P<0.05).

The above-described and other features will be appreciated and understood by those skilled in the art from the following detailed description, drawings, and appended claims.

### DETAILED DESCRIPTION

Disclosed herein are compositions and methods for reducing inflammation associated with atherosclerosis and/or vascular inflammatory diseases. Also provided herein are methods for treating or preventing atherosclerosis and/or vascular inflammation. Further provided are compositions comprising a S1 P₂ receptor antagonist and a pharmaceutically acceptable excipient.

The inventors have demonstrated that sphingosine 1-phosphate receptor-2 (S1P₂) inhibitors block atherosclerosis and vascular cytokine expression (IL-1β,IL-18) in mouse models. In addition, it was shown that S1 P 2 inhibitors block cytokine expression by regulation of expression of the inflammasome caspase-11. Thus, S1P₂ inhibitors and caspase-11 inhibitors can be used to reduce inflammation in atherosclerosis and other diseases caused by vascular inflammation such as heart disease, stroke, peripheral vascular disease, vasculitis, and others.

Sphingosine-1-phosphate (S1P) is a multifunctional lipid mediator that signals via the SIP family of G protein-coupled receptors (S1PR). SIP is known to regulate vascular maturation, permeability and angiogenesis. By example, SIP is known to be a stimulator of angiogenesis, i.e., new blood vessel growth. As used herein, the terms S1P2R, S1P₂R, S1P2 receptor and S1P₂ receptor are used interchangeably to mean the sphingosine-1-phosphate receptor 2.

Inhibitors of cholesterol synthesis such as statins are used widely to treat atherosclerosis in humans. However, in atherosclerosis, both cholesterol and sphingomyelin are elevated. Currently there are no drugs available to control sphingomyelin and related metabolites such as sphingosine-1-phosphate.

Atherosclerotic vascular disease that leads to heart attacks and strokes remains a major cause of morbidity and mortality worldwide. Early atherosclerotic plaque is characterized by the deposition of lipoprotein-derived cholesterol and sphingolipids in the arterial wall, and the recruitment of monocytes into the subendothelial space. Within the plaque, monocyte-derived macrophages drive inflammation and lesion growth by secreting pro-inflammatory cytokines, including TNF-α and IL-1β. Although the ability of lipoprotein-derived cholesterol to initiate atheroma formation is well established, the role of sphingolipids in atherosclerosis and macrophage biology is not well understood. It is reported herein that the S1 P₂-receptor, a G protein-coupled receptor for the sphingolipid mediator SIP, is essential for atherosclerotic plaque development in vivo, and regulates the macrophage inflammasome, a multi-protein complex crucial for the release of the pro-atherosclerotic cytokines IL-1β and IL-18.

Using a mouse model of atherosclerosis, the inventors herein have demonstrated that the S1P₂ receptor promotes atherosclerosis by regulating macrophage expression of caspase-11, a key inflammasome component. Indeed, atherosclerotic lesions express inflammasome constituents in an S1P₂ receptor-dependent manner. Furthermore, S1P₂ receptor is required for caspase-11-containing inflammasome formation and the secretion of IL-1β and IL-18 in vivo. S1P₂ receptor regulation of inflammasome-specific caspase-11 provides a novel mechanistic link between sphingolipid signaling, innate immune function, and atherosclerosis. S1P₂ receptor/caspase-11 inhibition constitutes a novel strategy to combat atherosclerotic vascular disease.

In one embodiment, treating atherosclerosis includes inhibiting or reducing risk of cardiovascular and cerebrovascular diseases resulting from atherosclerosis, such as cardiac and/or cerebral ischemia, myocardial infarction, angina, peripheral vascular disease and stroke.

In one embodiment, a method of reducing inflammation associated with atherosclerosis and/or vascular inflammatory diseases comprises administering to an individual in need thereof an effective amount of an S1 P₂ receptor antagonist or caspase-11 antagonist. As used herein, the term administering includes administration to an individual suffering from atherosclerosis and/or vascular inflammation and administration preventatively or prophylactically to an individual at risk of atherosclerosis and/or vascular inflammation. Administration to an individual at risk of atherosclerosis and/or vascular inflammation can prevent atherosclerosis and/or vascular inflammation. In one embodiment, the individual is at risk of, or has been diagnosed with, atherosclerosis and/or vascular inflammation.

The terms “blocker”, “inhibitor”, or “antagonist” are used interchangeably to mean a substance that retards or prevents a chemical or physiological reaction or response. Exemplary blockers or inhibitors comprise, but are not limited to, antisense molecules, siRNA molecules, antibodies, small molecule antagonists and their derivatives. An S1P₂ receptor blocker or inhibitor inhibits the activity and/or concentration of an S1P₂ receptor. An S1P₂ receptor blocker or inhibitor is an S1P₂ receptor antagonist such as a small molecule, an antibody, an antisense nucleic acid or an siRNA.

In one embodiment, the S1P₂ receptor antagonist is a small molecule such as a molecule of Formula I:

Ar²—X [symbol] Y [symbol] Z—W—Ar¹ Formula I

wherein

Ar¹ is optionally substituted heterocycle or aromatic heterocycle;

Ar² is optionally substituted heterocycle or aromatic heterocycle;

W is NR^{a}—, O, or —CH₂—, wherein R^{a} is hydrogen or C₁-C₃ alkyl;

Z is —C(═O)—, —C(═S)—, O, —CH₂—, ═N—, or ═CH—;

Y is —NR^{a}—, —C(═O)—, —N═, —CH═, ═N—, or ═CH—; and

X is —NR^{a}—, —N═, —CH═, or —CH₂—. When substituted, the substituents on Ar¹ and Ar² include halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

Specifically, exemplary antagonists include those of Formula II wherein

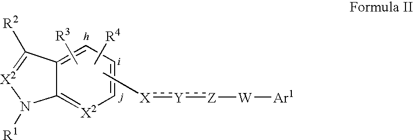

Ar¹ is aromatic heterocycle;

W, Z, Y and X are as previously defined;

R¹ is C₁-C₁₂ alkyl;

R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;

R³ and R⁴ can be positioned at h, i, or j, but not simultaneously at the same position; and

X² is N or —CR^{b}— wherein R^{b} is hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

More specifically, exemplary antagonists include those of Formula III wherein

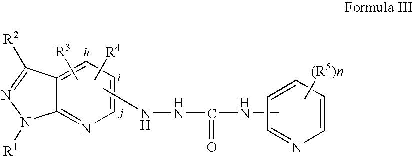

R¹, R², R³, and R⁴ are as previously defined;

each instance of R⁵ is halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy; and

n is 0, 1, 2, 3, or 4.

In a specific embodiment, antagonists include those of Formula III wherein R¹ is C₁-C₃ alkyl; R² is C₁-C₃ alkyl; R³ is at position h and is C₁-C₆ alkyl; R⁴ is hydrogen; R⁵ is halogen; and n is 2.

Additional exemplary antagonists include 1-[1,3-dimethyl-4-(2-methylethyl)-1H-pyrazolo[3,4-b]pyridin-6-yl]-4-(3,5-dichloro-4-pyridinyl)-semicarbazide (“JTE 013”; CAS No. [547756-93-4]); 1-[1,3-dimethyl-4-(2-methylethyl)-1H-pyrazolo[3,4-b]pyridin-6-yl]-4-(3,5-difluoro-4-pyridinyl)-semicarbazide; 1-[1,3-dimethyl-4-(2-methylethyl)-1H-pyrazolo[3,4-b]pyridin-6-yl]-4-(3,5-dibromo-4-pyridinyl)-semicarbazide; 1-[1,3-dimethyl-4-(2-methylethyl)-1H-pyrazolo[3,4-b]pyridin-6-yl]-4-(3,5-dichloro-4-phenyl)-semicarbazide; 1-[1,3-dimethyl-4-(2-methylethyl)-1H-pyrazolo[3,4-b]pyridin-6-yl]-4-(3,5-difluoro-4-phenyl)-semicarbazide; 1-[1,3-dimethyl-4-(2-methylethyl)-1H-pyrazolo[3,4-b]pyridin-6-yl]-4-(3,5-dibromo-4-phenyl)-semicarbazide; 1-[1,3-dimethyl-4-(2-methylethyl)-1H-pyrazolo[3,4-b]pyridin-6-yl]-4-(3,5-dimethoxy-4-pyridinyl)-semicarbazide; 1-[1,3-dimethyl-4-(2-methylethyl)-1H-pyrazolo[3,4-b]pyridin-6-yl]-4-(3,5-dimethyl-4-pyridinyl)-semicarbazide; 1-[1,3-dimethyl-4-(2-methylethyl)-1H-pyrazolo[3,4-b]pyridin-6-yl]-4-(3-chloro, 5-fluoro-4-pyridinyl)-semicarbazide; 1-[1,3-dimethyl-4-(2-methylethyl)-1H-pyrazolo[3,4-b]pyridin-6-yl]-4-(3,5-dimethoxy-4-phenyl)-semicarbazide; 1-[1,3-dimethyl-4-(2-methylethyl)-1H-pyrazolo[3,4-b]pyridin-6-yl]-4-(3,5-dimethyl-4-phenyl)-semicarbazide; 1-[1,3-dimethyl-4-(2-methylethyl)-1H-pyrazolo[3,4-b]pyridin-6-yl]-4-(3-chloro, 5-fluoro-4-phenyl)-semicarbazide.

Exemplary antagonists include the pyrazolopyridine and related compounds disclosed in WO 01/98301 to Kawasaki et al., incorporated herein by reference in its entirety.

The active agents can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment, an S1P₂ receptor or caspase-11 inhibitor is an antibody. The present disclosure includes isolated (i.e., removed from their natural milieu) antibodies that selectively bind an S1P₂ receptor. As used herein, the term “selectively binds to” refers to the ability of antibodies of the present disclosure to preferentially bind to an S1P₂ receptor or caspase-11. Binding can be measured using a variety of methods standard in the art including enzyme immunoassays (e.g., ELISA), immunoblot assays, and the like; see, for example, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999. An antibody selectively binds to or complexes with an S1P₂ receptor or caspase-11, preferably in such a way as to reduce the activity of an S1P₂ receptor or caspase-11.

As used herein, antibody includes antibodies in serum, or antibodies that have been purified to varying degrees, specifically at least about 25% homogeneity. The antibodies are specifically purified to at least about 50% homogeneity, more specifically at least about 75% homogeneity, and most specifically greater than about 90% homogeneity. Antibodies may be polyclonal antibodies, monoclonal antibodies, humanized or chimeric antibodies, anti-idiotypic antibodies, single chain antibodies, Fab fragments, fragments produced from an Fab expression library, epitope-binding fragments of the above, and the like. An antibody includes a biologically active fragment, that is, a fragment of a full-length antibody the same target as the full-length antibody. Biologically active fragments include Fab, F(ab′)₂ and Fab′ fragments.

Antibodies are prepared by immunizing an animal with full-length polypeptide or fragments thereof. The preparation of polyclonal antibodies is well known in the molecular biology art; see for example, Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology, (1992).

A monoclonal antibody composition is produced, for example, by clones of a single cell called a hybridoma that secretes or otherwise produces one kind of antibody molecule. Hybridoma cells are formed, for example, by fusing an antibody-producing cell and a myeloma cell or other self-perpetuating cell line. Numerous variations have been described for producing hybridoma cells.

In one embodiment, monoclonal antibodies are obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3X63-Ag8.653, Sp2/0-Ag14) and the like. Treatment with the transforming agent results in production of a hybridoma by means of fusing the suspended spleen cells with, for example, mouse myeloma cells. The transformed cells are then cloned, preferably to monoclonality. The cloning is performed in a medium that will not support non-transformed cells, but that will support transformed cells. The tissue culture medium of the cloned hybridoma is then assayed to detect the presence of secreted antibody molecules by antibody screening methods known in the art. The desired clonal cell lines are then selected.

A therapeutically useful antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions.

In addition, chimeric antibodies can be obtained by splicing the genes from a mouse antibody molecule with appropriate antigen specificity together with genes from a human antibody molecule of appropriate biological specificity. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies are used to produce single chain antibodies, as described, for example, in U.S. Pat. No. 4,946,778. Single chain antibodies are formed by linking the heavy and light chain fragments of the Fv region via an amino acid bridge, resulting in a single chain polypeptide.

In one embodiment, antibody fragments that recognize specific epitopes are generated by techniques well known in the art. Such fragments include Fab and F(ab′)₂ fragments produced by proteolytic digestion, and Fab′ fragments generated by reducing disulfide bridges. Fab, F(ab′)₂ and Fab′ fragments of antibodies can be prepared. Fab fragments are typically about 50 kDa, while F(ab′)₂ fragments are typically about 100 kDa in size. Antibodies are isolated (e.g., on protein G columns) and then digested and purified with sepharose coupled to papain and to pepsin in order to purify Fab and F(ab′)₂ fragments according to protocols provided by the manufacturer (Pierce Chemical Co.). The antibody fragments are further purified, isolated and tested using ELISA assays. Antibody fragments are assessed for the presence of light chain and Fc epitopes by ELISA.

In another embodiment, antibodies are produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic subjects are known in the art.

The structure of the antibody may also be altered by changing the biochemical characteristics of the constant regions of the antibody molecule to a form that is appropriate to the particular context of the antibody use. For example, the isotype of the antibody may be changed to an IgA form to make it compatible with oral administration. IgM, IgG, IgD, or IgE isoforms may have alternate values in the specific therapy in which the antibody is used.

Antibodies are purified by methods known in the art. Suitable methods for antibody purification include purification on Protein A or Protein G beads, protein chromatography methods (e.g., DEAE ion exchange chromatography, ammonium sulfate precipitation), antigen affinity chromatography and others.

In one embodiment, a monoclonal antibody that acts as an S1P₂ receptor inhibitor is formed using E. coli-derived S1P2 full length antigen to develop a murine monoclonal antibody as described in Oh et al., Journal of Biological Chemistry, pp. 9082-9089 (2007). The monoclonal antibody is purified from the hybridoma using protein-A sepharose. A monoclonal antibody against the S1P2 receptor is used alone or in combination with other S1P2 receptor inhibitors and regulating agents disclosed herein.

In one embodiment, the S1P₂ receptor or caspase-11 antagonist comprises an antisense RNA. An antisense RNA (aRNA) is single-stranded RNA that is complementary to a messenger RNA (mRNA) strand transcribed within a cell. Antisense RNA may be introduced into a cell to inhibit translation of a complementary mRNA by base pairing to it and physically obstructing the translation machinery. An antisense molecule specific for an S1P₂ receptor should generally be substantially identical to at least a portion, specifically at least about 20 continuous nucleotides, of the nucleic acid encoding the S1P₂ receptor, but need not be identical. The antisense nucleic acid molecule can be designed such that the inhibitory effect applies to other proteins within a family of genes exhibiting homology or substantial homology to the nucleic acid. The introduced antisense nucleic acid molecule also need not be full-length relative to either the primary transcription product or fully processed mRNA. Generally, higher homology can be used to compensate for the use of a shorter sequence. Furthermore, the antisense molecule need not have the same intron or exon pattern, and homology of non-coding segments will be equally effective. Antisense phosphorothioate oligodeoxynucleotides (PS-ODNs) is exemplary of an antisense molecule specific for the S1 P₂ receptor.

In another embodiment, the S1P₂ receptor or caspase-11 antagonist comprises an siRNA. RNA interference (“RNAi”) is a method of post-transcriptional gene regulation that is conserved throughout many eukaryotic organisms. RNAi is induced by short (i.e., less than 30 nucleotide) double stranded RNA (“dsRNA”) molecules, which are present in the cell. These short dsRNA molecules, called “short interfering RNA” or “siRNA,” cause the destruction of messenger RNAs (“mRNAs”), which share sequence homology with the siRNA to within one nucleotide resolution. Without being held to theory, it is believed that the siRNA and the targeted mRNA bind to an “RNA-induced silencing complex” or “RISC”, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore effective for inhibiting expression of a target gene.

siRNA comprises short double-stranded RNA of about 17 nucleotides to about 29 nucleotides in length, specifically about 19 to about 25 nucleotides in length, that are targeted to the target mRNA, that is, the S1P2 receptor. The siRNA comprise a sense RNA strand and a complementary antisense RNA strand annealed together by standard Watson-Crick base-pairing interactions (“base-paired”). The sense strand comprises a nucleic acid sequence which is identical to a target sequence contained within the target mRNA.

The sense and antisense strands of siRNA comprise two complementary, single-stranded RNA molecules, or comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules.

One or both strands of the siRNA can also comprise a 3′ overhang. A “3′ overhang” refers to at least one unpaired nucleotide extending from the 3′-end of a duplexed RNA strand. In one embodiment, the siRNA comprises at least one 3′ overhang of 1 to about 6 nucleotides (which includes ribonucleotides or deoxynucleotides) in length, specifically of 1 to about 5 nucleotides in length, more specifically of 1 to about 4 nucleotides in length, and particularly specifically of about 2 to about 4 nucleotides in length. In the embodiment in which both strands of the siRNA molecule comprise a 3′ overhang, the length of the overhangs can be the same or different for each strand. In one embodiment, the 3′ overhang is present on both strands of the siRNA, and is 2 nucleotides in length. For example, each strand of the siRNA of the can comprise 3′ overhangs of dithymidylic acid (“TT”) or diuridylic acid (“uu”). In order to enhance the stability of the siRNA, the 3′ overhangs can also be stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′;-deoxythymidine significantly enhances the nuclease resistance of the 3′ overhang in tissue culture medium.

The siRNA is obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. The siRNA expressed from recombinant plasmids is isolated from cultured cell expression systems by standard techniques, or is expressed intracellularly at or near the area of neovascularization in vivo. The siRNA can also be expressed from recombinant viral vectors intracellularly at or near the area of neovascularization in vivo. The recombinant viral vectors comprise sequences encoding the siRNA and a promoter for expressing the siRNA sequences. Exemplary promoters include, for example, the U6 or H1 RNA pol III promoter sequences and the cytomegalovirus promoter.

One skilled in the art can readily determine an effective amount of the siRNA to be administered to a given subject, by taking into account factors such as the size and weight of the subject; the extent of the neovascularization or disease penetration; the age, health and sex of the subject; the route of administration; and whether the administration is regional or systemic. Generally, an effective amount of the siRNA comprises an intercellular concentration at or near the neovascularization site of about 1 nanomolar (nM) to about 100 nM, specifically about 2 nM to about 50 nM, more specifically about 2.5 nM to about 10 nM. It is contemplated that greater or lesser amounts of siRNA can be administered.

The inventors herein investigated the role of S1P signaling in atherosclerosis and/or vascular inflammation. Disclosed herein are methods of treatment comprising administering to a subject an effective amount of an S1 P₂ receptor antagonist. In one aspect, the agent is substantially purified (e.g., substantially free from substances that limit its effect or produce undesired side-effects). The subject is specifically an animal, e.g., such as cows, pigs, horses, chickens, cats, dogs, etc., and is more specifically a mammal, and most specifically a human.

Pharmaceutical compositions include a therapeutically effective amount of an active agent with a pharmaceutically acceptable excipient. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin. In one embodiment, the composition is formulated in accordance with routine procedures as a pharmaceutical composition adapted for topical administration to human beings. Such pharmaceutical compositions are liquid, gel, ointment, salve, slow release formulations or other formulations suitable for ophthalmic administration.

In various embodiments, compositions comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

For oral administration, the pharmaceutical preparation can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art.

Preparations for oral administration can be suitably formulated to give controlled release of the active compound.

For buccal administration, the compositions can take the form of tablets or lozenges formulated in conventional manner.

For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion via either intravenous, intraperitoneal or subcutaneous injection. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compositions can be formulated into creams, lotions, ointments or tinctures, e.g., containing conventional bases, such as hydrocarbons, petrolatum, lanolin, waxes, glycerin, or alcohol. The compositions can also be formulated in rectal compositions such as suppositories or retention enemas, e.g., containing conventional suppository bases such as cocoa butter or other glycerides.

In addition to the formulations described previously, the compositions can also be formulated as a depot preparation. Such long acting formulations can be administered by implantation (e.g., subcutaneously or intramuscularly) or by intramuscular injection. Thus, for example, the compositions can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration.

The amount of the S1P2 receptor or caspase-11 antagonist that may be combined with pharmaceutically acceptable excipients to produce a single dosage form will vary depending upon the host treated and the particular mode of administration. The specific therapeutically effective amount for a particular patient will depend on a variety of factors including the activity of the specific compound employed, the age, body weight, general health, sex, diet, time of administration, route of administration, rate of excretion, drug combination, and the severity of the particular disease undergoing therapy. In some instances, dosage levels below the lower limit of the aforesaid range may be more than adequate, while in other cases still larger doses may be employed without causing any harmful side effects provided that such higher dose levels are first divided into several small doses for administration throughout the day. The concentrations of the compounds described herein found in therapeutic compositions will vary depending upon a number of factors, including the dosage of the drug to be administered, the chemical characteristics (e.g., hydrophobicity) of the compounds employed, and the route of administration. The preferred dosage of drug to be administered is likely to depend on such variables as the type and extent of progression of the disease or disorder, the overall health status of the particular patient, the relative biological efficacy of the compound selected, and formulation of the compound excipient, and its route of administration, as well as other factors, including bioavailability, which is in turn influenced by several factors.

In various embodiments, the S1P₂ receptor or caspase-11 antagonists may be administered in combination with one or more additional compounds or therapies or medical procedures.

The invention is illustrated by the following non-limiting examples.

### EXAMPLES

### Materials and Methods

### Methods Summary

C57BL/6x129Sv mice with targeted disruption of the S1p2 gene were generated as previously reported. Mice were maintained on a mixed C57BL/6 and 129Sv genetic background before crossing with Apoe^{-/-} mice, purchased from Jackson Laboratories (Bar Harbor, Me.). All experiments were performed with s1p2^{-/-} and s1p2^{+/+} littermate controls. All procedures involving mice were approved by the University of Connecticut Health Center Animal Care Committee. For atherosclerosis development, mice were fed a high cholesterol diet (TD88137) and analyzed en face after 13 weeks. Longitudinal preparations of fixed aortic arch and abdominal aorta were pinned on black wax and stained with Oil Red O (ORO). Image analysis and quantification was performed with Image Pro Plus Analysis Software (Media Cybernetics). For the aortic root analysis, cryosections (7 μm thick) throughout the aortic sinus were collected and stained for Hematoxylin Phloxin and Saffron (HPS), Trichrome and Oil Red O. Antibodies used were: rat-anti-MOMA-2 (Serotec), rabbit polyclonal anti-S1P₂ receptor (1:200), mouse-anti-α smooth muscle actin (Sigma), rabbit anti-Caspase-1 (Santa Cruz) and rat anti-Caspase-11 (Sigma). Images of the sections were observed with Zeiss Axioscop Microscope for transmitted-light brightfield; acquired and analysed with digital image processing software Axiovision. Fluorescent images were acquired with a Zeiss LSM 510 META for confocal light microscopy and analysed with Zeiss LSM Image Browser Software. For bone marrow transplantation, male Apoe^{-/-} mice were lethally irradiated with two 550 rad doses, 4 hours apart, from a ¹³⁷C source (Gammacell-40, MDS Nordion, Kanata, Canada). Recipient mice were reconstituted, via the lateral tail vein, with 5×10⁶ unfractionated bone marrow cells from Apoe^{-/-}s1p2^{-/-} and Apoe^{-/-}s1p2^{+/+} donors. Recipients were maintained on normal chow for 4 weeks, then placed on a high fat diet for 13 weeks and analyzed for lesion development.

Isolation of Thioglycolate-Elicited Peritoneal Cells. Mice were injected i.p. with 2 ml of 3% thioglycolate. Four days later, the peritoneal fluid was collected, and cells monolayer was prepared by addition of 1 ml/well (2×10⁶ macrophages) into 35 mm culture dishes. Macrophage adhesion was allowed to proceed for 4 h at 37° C. in a 5% CO₂ atmosphere.

Foam cell formation. Bone Marrow Derived Macrophages were plated on round glass coverslip in DMEM supplemented with 10% FBS and antibiotics. The next day, cells were treated with oxLDL (50 ng/ml) for 3 hrs, 5 hrs and 24 hrs at 37° C., followed by staining with Oil Red O and mounting with aqueous mounting medium. Images of random fields were captured and analysed with Image Pro Plus Analysis Software (Media Cybernetics).

RNA isolation. RNA was extracted (RNeasy kit; Qiagen) from mouse aortas or with RNAstat-60 (Tel-Test. B, Friendswood, Tex.) for macrophages. First-strand cDNA was synthesized using random hexamers, murine leukemia virus reverse transcriptase and accompanying reagents (Invitrogen Corp.) for 1 hr at 37° C. Mouse RT-PCR primers shown in Table 1 were designed with Primer Express software (Applied Biosystems). Amplification and data analysis was performed in ABI Prism 7900HT Sequence Detection System (Applied Biosystems). Messenger RNA (mRNA) levels were quantified and corrected for mGAPDH.

Immunoprecipitation experiments. Cell or spleen extracts were prepared with RIPA buffer (25 mM Tris-HCl pH 7.6, 150 mM NaCl, 1% NP-40, 1% sodium deoxycholate, 0.1% SDS). Caspase-1 was immunoprecipitated with polyclonal rabbit anti-caspase-1 antibody (Santa Cruz) crosslinked to protein A beads and Caspase-11 was immunoprecipitated with monoclonal rat anti-caspase-11 antibody, crosslinked to protein G beads. For western blot analysis, BMDM were treated with LPS (1 μg/ml, E. coli serotype 026:B6; Sigma). Aortas or cells were solubilized in RIPA buffer or 2×SDS-sample buffer (20 mM DTT, 6% SDS, 0.25 M Tris pH 6.8, 10% Glycerol, bromophenyl blue, protease inhibitors, 1 mM sodium orthovanadate and 1 mM NaF), sonicated, boiled and separated by SDS-PAGE gel electrophoresis. Membranes were incubated with the following antibodies: anti-b-actin (Sigma), anti-eNOS (BD Pharmingen), anti-COX-2 (Cayman), anti-Caspase-1 (Santa Cruz), anti-Caspase-11 (Sigma), anti-IκBα, anti-VCAM (Santa Cruz), anti-phospho p38, anti-phospho ERK, p38 and ERK (Cell Signaling). Cells were treated as indicated with 1 μg/ml LPS (Sigma) and 50 ng/ml TNFα (Sigma) Immunoreactive bands density was quantified with Image Pro Plus Analysis Software (Media Cybernetics).

Cytokine measurements. Lipopolysaccharide (LPS) (40 mg/kg, E. coli serotype 0127:B8; Sigma) was intraperitoneally injected into WT and KO animals. After 3 hrs or 5 hrs treatment, animals were anesthetized and blood was collected by final cardiac puncture. Mouse serum levels of IL-113, IL-18 (Invitrogen) and TNFα, IFNγ (e-Bioscience) were measured by Enzyme Linked ImmunoSorbent assay (ELISA).

Statistics. All data are expressed as mean±SEM. A 2-tailed Student's t Test was used for statistical analysis. P values less than 0.05 were considered significant.

### Examples

### Example 1

### Requirement of S1P₂ Receptor in Atherosclerosis

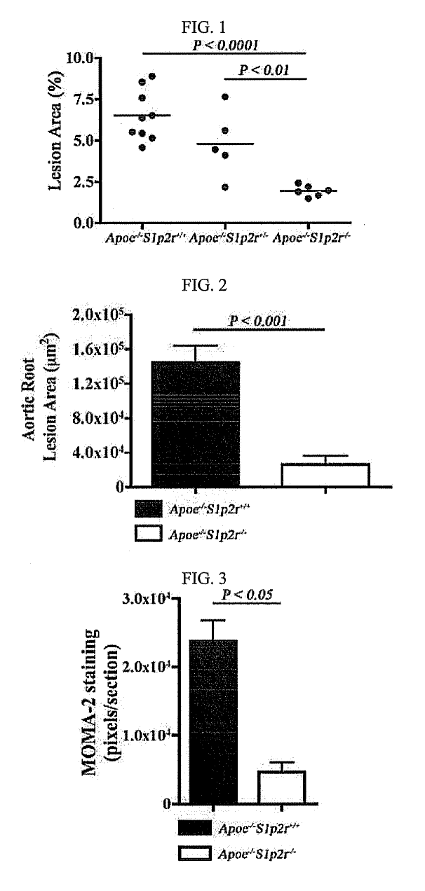

To investigate the role of the S1P₂ receptor in atherosclerosis, Apoe^{-/-}S1p2r^{-/-} mice and Apoe^{-/-}S1p2r^{+/+} littermate controls were placed on a high fat “Western” diet for 13 weeks. Mouse aortae were prepared and stained with Oil Red O to highlight the plaques of aortic arch, thoracic and abdominal aorta (data not shown). En face analysis of atheromatous lesions revealed significant, uniform and gene dosage-dependent reduction (>70% in all areas of the aortic tree) in Apoe^{-/-}S1p2r^{-/-} mice compared to Apoe^{-/-}S1p2r^{+/+} or Apoe^{-/-}S1p2r^{+/-} littermate controls (FIG. 1). Hematoxylin-Phloxin-Saffron (HPS), Trichrome, and Oil Red O staining of serial cross-sections from the aortic sinus was performed to examine the presence of fibrous caps, connective tissue content and lipid deposition. As shown in FIG. 2, loss of S1P₂ receptor led to a marked reduction (>80%) in atheromatous plaque area (fibrous tissue and collagen content) as well as lipid deposition. Plaques from the Apoe^{-/-}S1p2r^{-/-} mice had significantly decreased anuclear and afibrotic area. Furthermore, macrophage infiltration in the vessel wall was examined by immunostaining with a macrophage/monocyte specific antibody (MOMA-2). Apoe^{-/-}S1p2r^{-/-} mice showed markedly diminished (>80% reduction) macrophage infiltration compared to the Apoe^{-/-}S1p2r^{+/+} counterparts (FIG. 3). In addition, body weight, cholesterol, triglyceride and S1P (data not shown) levels were not different between Apoe^{-/-}S1p2r^{-/-} mice and littermate controls, indicating that sterol and triglyceride metabolism are unlikely mediators of S1P₂ receptor-induced atherosclerosis (data not shown). These data suggest a novel role for S1P₂ receptor as a major regulator of atherosclerotic plaque development.

### Example 2

### Requirement of S1P₂ Receptor in Myeloid Cells for Atheroma Development

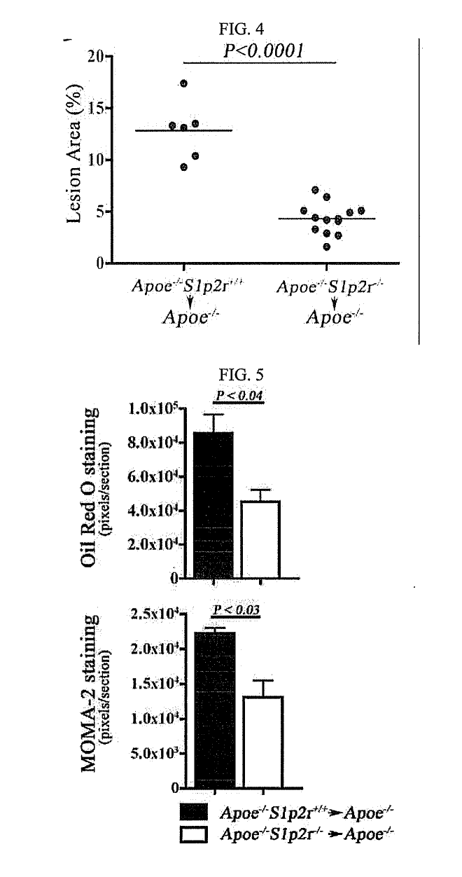

Given that macrophages play major roles in driving plaque inflammation and progression, the role of S1P₂ receptor in the hematopoietic compartment was examined. Immunostaining for the S1P₂ receptor in the aortic sinus showed that S1P₂ is expressed in atherosclerotic plaques, in cells that resemble macrophage-like foam cells (data not shown). Indeed, bone marrow derived macrophages (BMDM) express high levels of S1P₂ receptor mRNA in addition to S1P₁, which was shown to inhibit inflammatory events. Lack of S1P₂ did not change the expression levels of S1P₁, S1P₃ and S1P₄ receptor transcripts whereas S1P₅ receptor expression is undetectable (data not shown). Bone marrow chimeras were generated by transplanting lethally irradiated Apoe^{-/-} mice with Apoe^{-/-}S1p2r^{+/+} or Apoe^{-/-}S1p2r^{-/-} bone marrow. After 13 weeks on a “western” diet, en face analysis demonstrated a significant reduction in atherosclerotic lesion area throughout the aorta (˜65%) in mice receiving Apoe^{-/-}S1p2r^{-/-} marrow compared to mice that received Apoe^{-/-}S1p2r^{+/+} marrow (FIG. 4). Analysis of circulating peripheral blood monocytes (CD11b⁺, CD115⁺), polymorphonuclear leukocytes (CD11b⁺, CD115⁻, Gr1⁺), and CD4⁺, CD8⁺, or B220⁺lymphocytes revealed no differences between Apoe^{-/-}S1p2r^{+/+} and Apoe^{-/-}S1p2r^{-/-} mice (data not shown). Atherosclerotic lesions at the aortic root showed decreased lipid and macrophage accumulation (FIG. 5), suggesting that the S1P₂ receptor in macrophages is sufficient to promote atherosclerosis.

### Example 3

### S1P₂ Receptor Regulates Caspase-11 Expression

Lipid-laden macrophage (foam cells) are a major component of atherosclerotic plaques and result from the uptake of modified lipoproteins. To examine a role for S1P₂ in foam cell formation, s1p2^{-/-} and control s1p2^{+/+} BMDM were treated with oxidized LDL (oxLDL) (50 μg/ml), followed by staining with Oil Red O to visualize lipid accumulation. No differences in foam cell formation in BMDM lacking S1P₂ (data not shown) were detected, suggesting that S1P₂ receptor is dispensable for foam cell differentiation.

Lipid-laden macrophage (foam cells) is a major component of atherosclerotic plaques and result from the uptake of modified lipoproteins. In addition, oxidized low density lipoproteins (oxLDL) activate the macrophages via several receptors including CD36 and toll-like receptor-4 (TLR4). To identify novel downstream effectors of the S1P₂ receptor, S1p2r^{+/+} and S1p2r^{-/-} BMDM were treated with oxLDL (50 μg/ml) to induce foam cell formation and the transcriptome changes were defined using the Illumina™ microarray (Illumina, San Diego, Calif.). Foam cell differentiation was not altered in S1p2r^{+/+} and S1p2r^{-/-} cells (data not shown). Interestingly caspase-11 mRNA was one of the most down regulated transcripts in S1p2r^{-/-} foam cells. Mouse caspase-1, -11, and -12 constitute the subfamily of proinflammatory caspases that regulate cytokine maturation, apoptosis and leukocyte migration. Caspase-1 and -11 are also components of the inflammasome, a cytoplasmic multiprotein complex that translates various extracellular stimuli (microbial epitopes and patterns, uric acid crystals, alumina adjuvant) into an inflammatory output such as the secretion of signal peptide-less cytokines IL-113, IL-18 and IL-33. Little is known about the role of inflammatory caspases in atherogenesis.

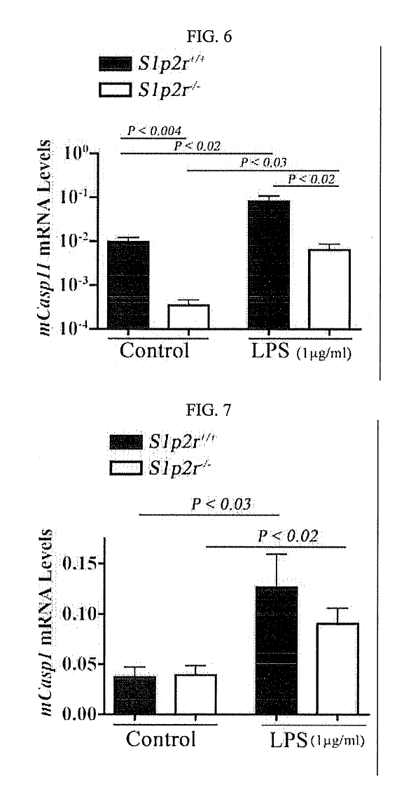

The mechanism of regulation of caspase-11 by the S1P₂ receptor was next studied. Quantitative RT-PCR analysis showed that S1p2r^{-/-} BMDM had significantly lower expression of caspase-11 mRNA (>25 fold reduction) compared to littermate controls (FIG. 6), suggesting that S1P₂ receptor regulates the expression of caspase-11. Upon TLR4 stimulation with lipopolysaccharide (LPS) (11.1 g/ml), caspase-11 gene expression was induced in both wild-type and knockout BMDM; however, expression level of caspase-11 mRNA in S1p2r^{-/-} macrophages was significantly attenuated (>10 fold) compared to S1p2r^{+/+} cells (FIG. 6). In contrast, caspase-1 transcript was induced to a similar extent (FIG. 7). LPS treatment induced equivalent kinetics and magnitude of signaling events (p38 stress activated protein kinase and p42/44 ERK activation, IκBα degradation, caspase-3, TNF-α and IL-1β mRNA expression) in wild-type and knock-out BMDM cells (data not shown), suggesting that caspase-11 is a selective transcriptional target of S1P₂ signaling in macrophages.

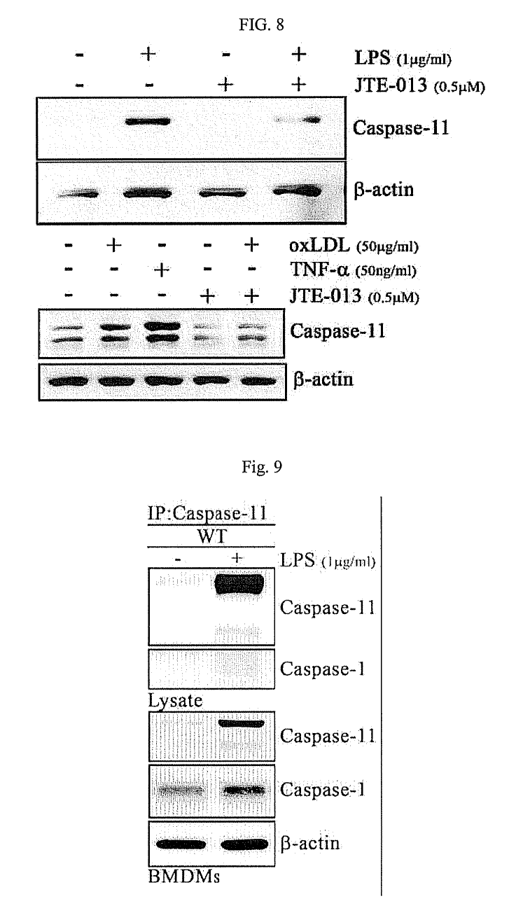

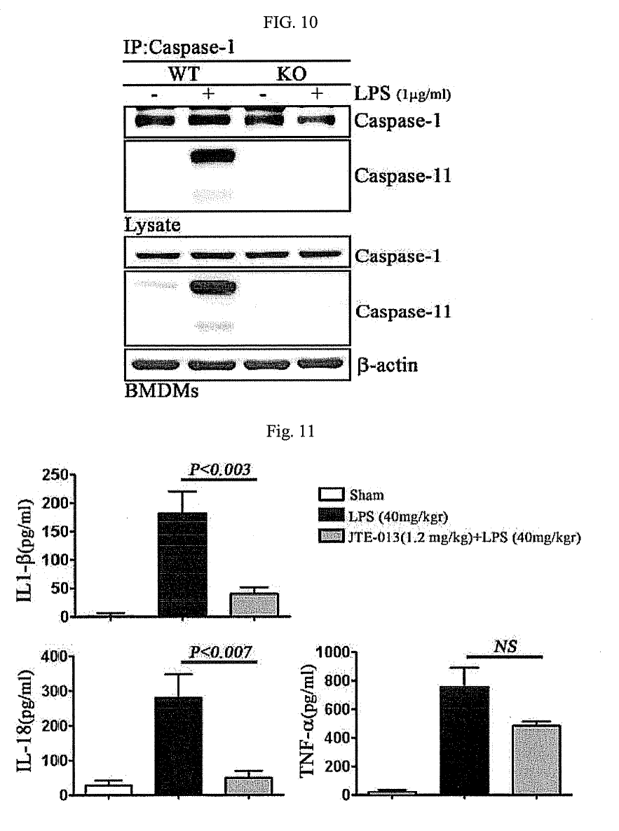

In S1p2r^{+/+} macrophages, caspase-11 protein expression was low under basal conditions, but was strongly induced upon LPS or TNF-a stimulation. In sharp contrast, LPS or TNF-a treatment failed to induce caspase-11 protein in S1p2r^{-/-} macrophages (data not shown). Since the difference in protein expression between wild-type and knockout cells is more pronounced than the transcript levels, the S1P₂ receptor may exert an additional post-transcriptional control over caspase-11 in addition to the NF-κB-dependent transcriptional activation. However, NFκB activation was equivalent between S1p2r^{+/+} and S1p2r^{-/-} cells, as measured by COX-2 expression (a NFκB-response gene) or by IκBα degradation (data not shown). In addition, we could not reverse the caspase-11 null phenotype of S1p2r^{-/-} macrophage by the proteasome inhibitor MG132 (1-20 μM) or the lysosomotropic agent chloroquine (10-100 μM) (data not shown), suggesting that the receptor does not inhibit caspase-11 protein degradation. However, in mouse embryonic fibroblasts (MEF), adenoviral-mediated expression of S1P₂ receptor induced caspase-11 polypeptide, suggesting that S1P₂ receptor directly regulates caspase-11 expression (data not shown). Blocking the S1P₂ receptor with JTE-013 (500 nM) a specific pharmacological antagonist significantly reduced caspase-11 polypeptide expression induced by LPS in mouse elicited peritoneal macrophages (FIG. 8). In view of the requirement for the S1P₂ receptor in atherosclerotic plaque development, we assessed if pro-atherosclerotic lipoproteins induce caspase-11 expression. Indeed, oxidized-LDL (50 μg/ml) induced caspase-11 protein expression in mouse elicited peritoneal macrophages (FIG. 8). The effect of oxLDL was blocked by JTE-013, suggesting the requirement of S1P₂ receptor function for caspase-11 induction.

### Example 4

### S1P₂ Receptor Modulates Inflammasome Complex Formation and Pro-Inflammatory Cytokines Production

Furthermore, to test whether the lack of S1P₂ receptor affects the inflammasome complex formation, reciprocal co-immunoprecipitation experiments were conducted. In LPS treated BMDM, caspase-11 is present as a complex with caspase-1 in S1p2r^{+/+} but not in S1p2r^{-/-} cells (FIG. 9, 10). These data suggest that S1P₂ receptor function in macrophages regulates inflammasome complex formation by selectively regulating caspase-11 expression. Since the S1P₂ receptor is required for caspase-11 driven inflammasome activation and inflammasome complexes are involved in the cellular processing of stress signals into signal-less cytokine release, the hypothesis that S1 P₂ receptor pathway regulates pro-inflammatory cytokine production was tested. Indeed, LPS induced production of IL-1β and IL-18 cytokines in vivo was significantly reduced in animals treated with the S1P₂ receptor antagonist, JTE-013. TNF-α cytokine that is not an inflammasome substrate was not significantly altered (FIG. 11).

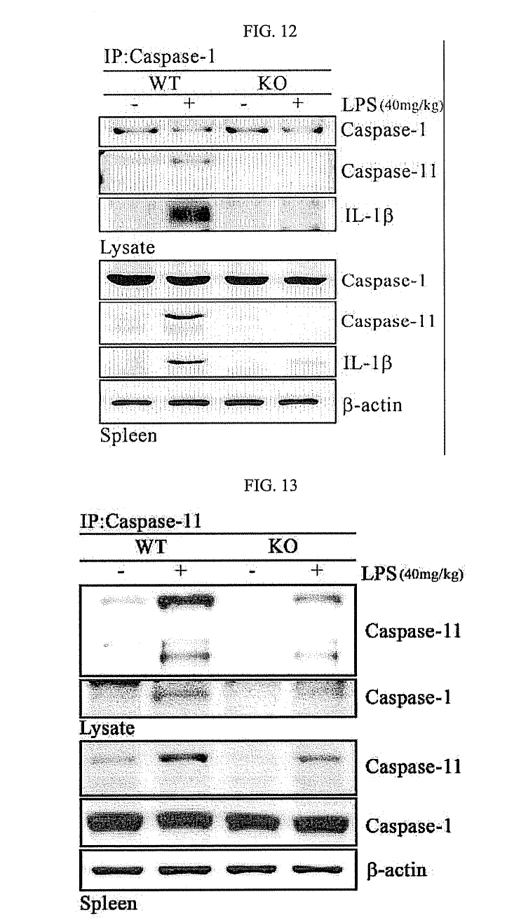

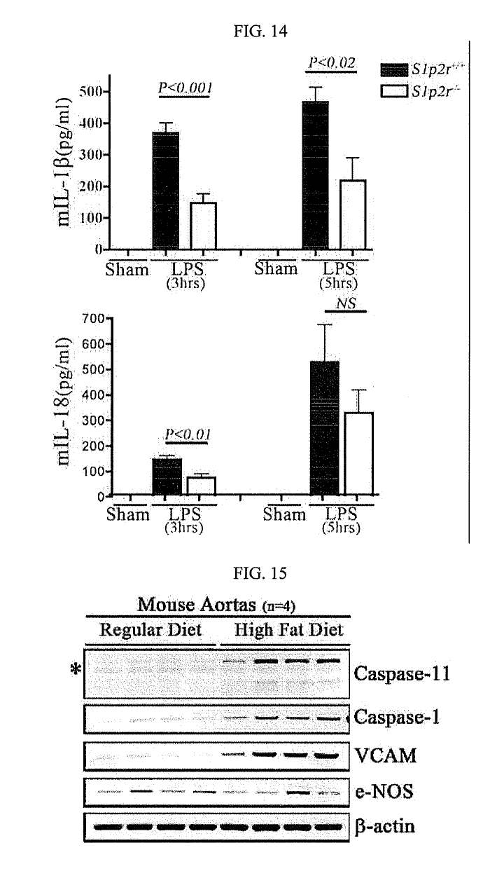

Moreover, spleen extracts from wild-type and LPS-treated mice were immunoprecipitated with caspase antibodies. Caspase-1 and -11 are found in a complex with IL-1β in spleen extracts; LPS treatment significantly increased the levels of IL-1β and caspase-11 in the immunoprecipitates. This was attenuated significantly in S1p2r^{-/-} extracts (FIG. 12, 13). These data provide strong evidence for regulation of inflammasome function in vivo by the S1P₂ receptor pathway. In agreement with the results obtained with the JTE-013 antagonist, S1p2r^{-/-} mice had significantly reduced serum IL-1β and IL-18 levels compared to control S1p2r^{+/+} mice (FIG. 14). In addition, we did not observe differences in serum TNF-a or IFNγ, which contain signal peptide sequences and are secreted via the classical secretory pathway (data not shown). These in vivo data strongly suggest that S1P₂ receptor regulation of caspase-11 regulates signal peptide-less, proatherosclerotic cytokine processing and release.

### Example 5

### Inflammatory Caspases are Expressed in Atheromatic Lesions and Caspase-11 Promotes Atherogenesis Similar to S1P₂ Receptor Proatherogenic Role

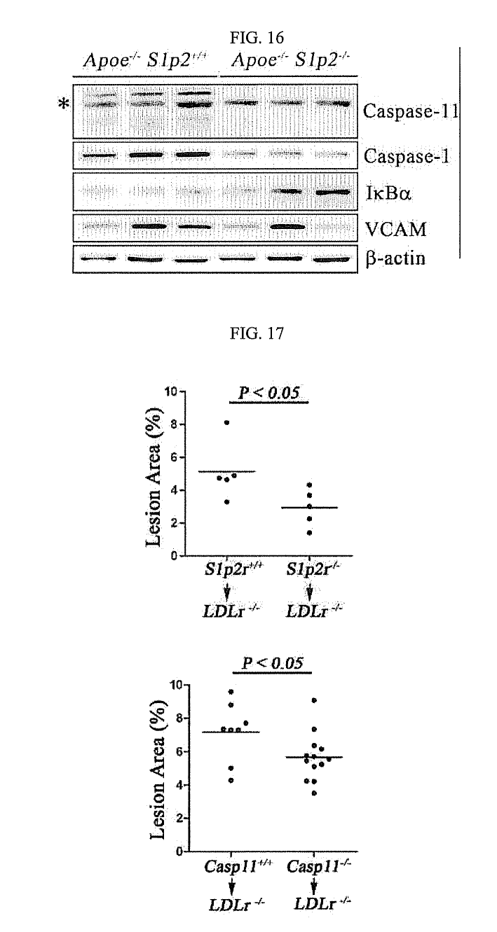

Next, the hypothesis that S1P₂ receptor/caspase-11 axis regulates pro-atherosclerotic cytokine expression in Apoe^{-/-} model of murine atherosclerosis was tested. Upon 13 weeks of high fat feeding of Apoe^{-/-} mice, inflammatory caspases (caspase-11 and -1) are strongly induced in protein extracts of mouse aortae. VCAM-1 expression, an indicator of inflamed endothelial cells, was also induced concomitantly whereas eNOS expression was not (FIG. 15). In sharp contrast, Apoe^{-/-}S1p2r^{-/-} aortas display significantly reduced expression of these inflammasome constituents. IκBα expression was induced in S1p2r^{-/-} aortae, which suggests attenuated NFκB signaling in atheromatous tissues in vivo. Expression of the endothelial cell-specific gene VCAM-1, which mediates monocyte influx into the atheroma, was not altered significantly (FIG. 16) Immunofluorescence staining of atheromatous regions in the aorta followed by confocal microscopy indicates that atherosclerotic plaques express the inflammatory caspase-11 and caspase-1 (data not shown). This induction was attenuated in S1p2r^{-/-} aortae, indicating that S1P, receptor regulates caspase-11 and consequent inflammasome activation during atheroma development. In order to examine the direct role of caspase-11 in atherogenesis, we generated bone marrow chimeras by transplanting lethally irradiated LDLr^{-/-} mice with either S1p2r^{-/-} or Casp11^{-/-} bone marrow. In agreement with the results obtained by the Apoe^{-/-} model of atherosclerosis, en face analysis demonstrated a significant reduction in atherosclerotic lesion area throughout the aorta in mice receiving S1p2r^{-/-} marrow compared to mice that received S1p2r^{+/+} marrow (FIG. 17). More importantly, mice transplanted with Casp11^{-/-} bone marrow present significant decrease in lesion area compared to control animals, suggesting that inflammatory caspase-11 is a major contributor in atherosclerotic disease (FIG. 17). Taken these results together, we suggest that S1P₂ receptor promotes atherogenesis by regulating inflammatory caspase-11 expression and inflammasome activation that facilitates release of mature cytokines and atherosclerotic disease development.

TABLE 1

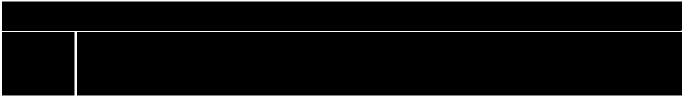

Body weight, cholesterol levels and triglycerides levels of Apoe^{-/-}s1p2^{+/+} (n=9) and Apoe^{-/-}s1p2^{-/-} (n=5) mice after 13 weeks on a high cholesterol diet.

TABLE 2
Primer sequences for quantitative real-time
RT-PCR

| Gene | Sequence of primers | |
|---|---|---|
| mS1p1 | F: ATGGTGTCCACTAGCATCCC | SEQ ID NO: 1 |
| | R: CGATGTTCAACTTGCCTGTGTAG | SEQ ID NO: 2 |
| mS1p2 | F: ATCGCCATCGAGAGACAAGT | SEQ ID NO: 3 |
| | R: AGACAATTCCAGCCCAGGAT | SEQ ID NO: 4 |
| mS1p3 | F: GCCTAGCGGGAGAGAAACCT | SEQ ID NO: 5 |
| | R: CCGACTGCGGGAAGAGTGT | SEQ ID NO: 6 |
| mS1p4 | F: GCCCTCATCCTAGTGGCTATC | SEQ ID NO: 7 |
| | R: GCCCAGACATTAGAACCAAAGA | SEQ ID NO: 8 |
| mS1p5 | F: TGTGCGCTCTATGCAAGGATT | SEQ ID NO: 9 |
| | R: CACGCTAAGGGTACGAAGCAG | SEQ ID NO: 10 |
| mCasp1 | F: TGGCATTAAGAAGGCCCATATAG | SEQ ID NO: 11 |
| | R: TGAGCCCCTGACAGGATGTC | SEQ ID NO: 12 |
| mCasp3 | F: GGTGGAGGCTGACTTCCTGTAT | SEQ ID NO: 13 |
| | R: CGACCCGTCCTTTGAATTTC | SEQ ID NO: 14 |
| mCasp11 | F: TGTTCCCCTGAAGAGTTCACAA | SEQ ID NO: 15 |
| | R: TTTCGTGTACGGCCATTGG | SEQ ID NO: 16 |
| mTNF-a | F: GGGCCACCACGCTCTTCTGTCT | SEQ ID NO: 17 |
| | R: GCCACTCCAGCTGCTCCTCCAC | SEQ ID NO: 18 |
| mIL-1b | F: TGGCCACCTTGTTCAGCTACG | SEQ ID NO: 19 |
| | R: GCCAAGGCCAAACACAGCATAC | SEQ ID NO: 20 |
| mGAPDH | F: CAACTACATGGTCTACATGTTCCAGT | SEQ ID NO: 21 |
| | R: TGACCCGTTTGGCTCCA | SEQ ID NO: 22 |

The inventors herein have discovered that the S1P2 receptor and caspase-11 are novel targets for the prevention and/or treatment of atherosclerosis and/or vascular inflammation. Antagonists of the S1P2 receptor or caspase-11 are suitable for novel compositions and methods for atherosclerosis and/or vascular inflammation.

The terms “a” and “an” do not denote a limitation of quantity, but

rather denote the presence of at least one of the referenced item.

The term “or” means “and/or”.

The terms “comprising”, “having”, “including”, and “containing” are to be construed as open-ended terms (i.e., meaning “including, but not limited to”).

The endpoints of all ranges directed to the same component or property are inclusive and independently combinable.

Unless defined otherwise, technical and scientific terms used herein have the same meaning as is commonly understood by one of skill in the art to which this invention belongs.

“Alkyl” is a branched or straight chain saturated aliphatic hydrocarbon group, having the specified number of carbon atoms, generally from 1 to about 12 carbon atoms. The term C₁-C₄alkyl as used herein indicates an alkyl group having from 1 to about 4 carbon atoms. Other embodiments include alkyl groups having from 1 to 8 carbon atoms, 1 to 6 carbon atoms or from 1 to 2 carbon atoms, e.g., C₁-C₈ alkyl, C₁-C₆ alkyl, and C₁-C₂ alkyl.

“Alkoxy” indicates an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups include, for example, methoxy groups.

“Cycloalkyl” indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane. A bicyclic cycloalkyl” is a saturated bicyclic group having only carbon ring atoms. Bicycloalkyl groups have 7 to 12 carbon ring atoms. Examples of bicycloalkyl groups include s-endonorbornyl and carbamethylcyclopentane.

“Mono- and/or di-alkylamino” indicates secondary or tertiary alkyl amino groups, wherein the alkyl groups are as defined above and have the indicated number of carbon atoms. The point of attachment of the alkylamino group is on the nitrogen. The alkyl groups are independently chosen. Examples of mono- and di-alkylamino groups include ethylamino, dimethylamino, and methyl-propyl-amino.

The term “heterocycle” indicates a 5-6 membered saturated, partially unsaturated, or aromatic (“aromatic heterocycle”) ring containing from 1 to about 4 heteroatoms chosen from N, O, and S, with remaining ring atoms being carbon or a 7-10 membered bicyclic saturated, partially unsaturated, or aromatic heterocylic ring system containing at least 1 heteroatom in the two ring system chosen from N, O, and S and containing up to about 4 heteroatoms independently chosen from N, O, and S in each ring of the two ring system. Unless otherwise indicated, the heterocyclic ring may be attached to its pendant group at any heteroatom or carbon atom that results in a stable structure. When indicated the heterocyclic rings described herein may be substituted on carbon or on a nitrogen atom if the resulting compound is stable. A nitrogen atom in the heterocycle may optionally be quaternized. It is preferred that the total number of heteroatoms in a heterocyclic groups is not more than 4 and that the total number of S and O atoms in a heterocyclic group is not more than 2, more preferably not more than 1. Examples of heterocyclic groups include, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benzo[b]thiophenyl, isoquinolinyl, quinazolinyl, quinoxalinyl, thienyl, isoindolyl, dihydroisoindolyl, 5,6,7,8-tetrahydroisoquinoline, pyridinyl, pyrimidinyl, furanyl, thienyl, pyrrolyl, pyrazolyl, pyrrolidinyl, morpholinyl, piperazinyl, piperidinyl, pyrrolidinyl, phthalazinyl, oxazolyl, indolizinyl, indazolyl, benzothiazolyl, benzimidazolyl, benzofuranyl, benzoisoxolyl, dihydro-benzodioxinyl, oxadiazolyl, thiadiazolyl, triazolyl, tetrazolyl, oxazolopyridinyl, imidazopyridinyl, isothiazolyl, and naphthyridinyl.

“Halo” or “halogen” indicates fluoro, chloro, bromo, and iodo.

“Perhaloalkyl” as used herein refers to alkyl groups perhalogenated with fluoro, chloro, bromo, iodo, or a combination of the foregoing halogens.

A dash (“-”) that is not between two letters or symbols is used to indicate a point of attachment for a substituent. For example, —(CH₂)C₃-C₇cycloalkyl is attached through carbon of the methylene (CH₂) group. A dash with a broken line above it indicates the bond can either be a single or double bond.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All cited patents, patent applications, and other references are incorporated herein by reference in their entirety.

1. A method of reducing inflammation associated with atherosclerosis in a subject in need thereof, comprising
administering to the subject in need of a reduction in inflammation associated with atherosclerosis a pharmaceutically effective amount of an inhibitor of the activity of the S1P2 receptor or caspase-11.

2. The method of claim 1, wherein the inhibitor is an antisense RNA, an siRNA, an antibody, or a small molecule.

3. The method of claim 1, wherein the inhibitor is an antagonist of the activity of the S1P2 receptor and is a small molecule of Formula I:
Ar²—X [symbol] Y [symbol] Z—W—Ar¹ Formula I
wherein
Ar¹ is an optionally substituted heterocycle or aromatic heterocycle;
Ar² is an optionally substituted heterocycle or aromatic heterocycle;
W is —NR^{a}—, O, or —CH₂— wherein R^{a} is hydrogen or C₁-C₃ alkyl;
Z is —C(═O)—, —C(═S)—, O, —CH₂—, ═N—, or ═CH—;
Y is —NR^{a}—, —C(═O)—, —N═, —CH═, ═N—, or ═CH—; and
X is —NR^{a}—, —N═, —CH═, or —CH₂—.

4. The method of claim 3, wherein the antagonist is a small molecule of Formula II: 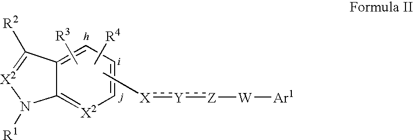
Ar¹ is an aromatic heterocycle;
R¹ is C₁-C₁₂ alkyl;
R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;
R³ and R⁴ are optionally positioned at h, i, or j, but not simultaneously at the same position; and
X² is N or —CR^{b}—, wherein R^{b} is hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

5. The method of claim 4, wherein the antagonist is a small molecule of Formula III: 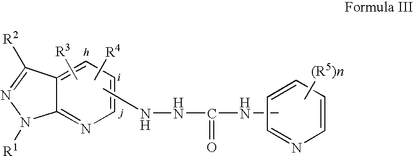
each instance of R⁵ is halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy; and
n is 0, 1, 2, 3, or 4.

6. The method of claim 5, wherein R¹ is C₁-C₃ alkyl; R² is C₁-C₃ alkyl, R³ is at position h and is C₁-C₆ alkyl; R⁴ is hydrogen; R⁵ is halogen; and n is 2.

7. A method of inhibiting or reducing a risk of cardiovascular and cerebrovascular diseases resulting from atherosclerosis in a subject in need thereof, comprising
administering to the subject a pharmaceutically effective amount of an inhibitor of the activity of the S1P2 receptor or caspase-11.

8. The method of claim 7, wherein the inhibitor is an antisense RNA, an siRNA, an antibody, or a small molecule.

9. The method of claim 8, wherein the inhibitor is an antagonist of the activity of the S1P2 receptor and is a small molecule of Formula I:
Ar²—X [symbol] Y [symbol] Z—W—Ar¹ Formula I
wherein
Ar¹ is an optionally substituted heterocycle or aromatic heterocycle;
Ar² is an optionally substituted heterocycle or aromatic heterocycle;
W is —NR^{a}—, O, or —CH₂— wherein R^{a} is hydrogen or C₁-C₃ alkyl;
Z is —C(═O)—, —C(═S)—, O, —CH₂—, ═N—, or ═CH—;
Y is —NR^{a}—, —C(═O)—, —N═, —CH═, ═N—, or ═CH—; and
X is —NR^{a}—, —N═, —CH═, or —CH₂—.

10. The method of claim 9, wherein the antagonist is a small molecule of Formula II: 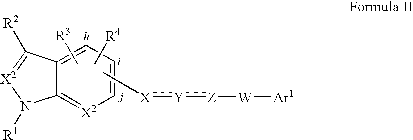
Arⁱ is an aromatic heterocycle;
R¹ is C₁-C₁₂ alkyl;
R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;
R³ and R⁴ are optionally positioned at h, i, or j, but not simultaneously at the same position; and
X² is N or —CR^{b}—, wherein R^{b} is hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

11. The method of claim 10, wherein the antagonist is a small molecule of Formula III: 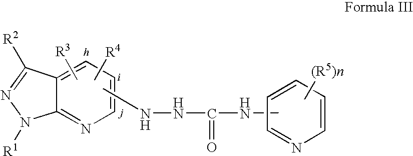
each instance of R⁵ is halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy; and
n is 0, 1, 2, 3, or 4.

12. The method of claim 11, wherein Rⁱ is C₁-C₃ alkyl; R² is C₁-C₃ alkyl, R³ is at position h and is C₁-C₆ alkyl; R⁴ is hydrogen; R⁵ is halogen; and n is 2.

13. The method of claim 7, wherein the cardiovascular and cerebrovascular disease resulting from atherosclerosis comprises cardiac and/or cerebral ischemia, myocardial infarction, angina, peripheral vascular disease or stroke.

14. A method of reducing inflammation associated with a vascular inflammatory disease in a subject in need thereof, comprising
administering to the subject in need of a reduction in inflammation associated with the vascular inflammatory disease a pharmaceutically effective amount of an inhibitor of the activity of the S1P2 receptor or caspase-11.

15. The method of claim 14, wherein the inhibitor is an antisense RNA, an siRNA, an antibody, or a small molecule.

16. The method of claim 15, wherein the inhibitor is an antagonist of the activity of the S1P2 receptor and is a small molecule of Formula I:
Ar²—X [symbol] Y [symbol] Z—W—Ar¹ Formula I
wherein
Ar¹ is an optionally substituted heterocycle or aromatic heterocycle;
Ar² is an optionally substituted heterocycle or aromatic heterocycle;
W is —NR^{a}—, O, or —CH₂— wherein R^{a} is hydrogen or C₁-C₃ alkyl;
Z is —C(═O)—, —C(═S)—, O, —CH₂—, ═N—, or ═CH—;
Y is —NR^{a}—, —C(═O)—, —N═, —CH═, ═N—, or ═CH—; and
X is —NR^{a}—, —N═, —CH═, or —CH₂—.

17. The method of claim 16, wherein the antagonist is a small molecule of Formula II: 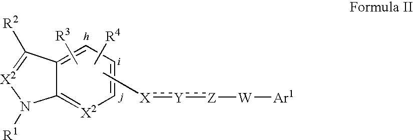
Arⁱ is an aromatic heterocycle;
R¹ is C₁-C₁₂ alkyl;
R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;
R³ and R⁴ are optionally positioned at h, i, or j, but not simultaneously at the same position; and
X² is N or —CR^{b}—, wherein R^{b} is hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

18. The method of claim 17, wherein the antagonist is a small molecule of Formula III: 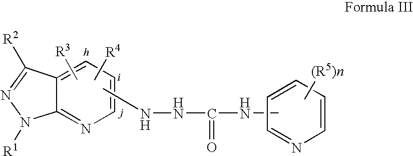
each instance of R⁵ is halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy; and
n is 0, 1, 2, 3, or 4.

19. The method of claim 18, wherein R¹ is C₁-C₃ alkyl; R² is C₁-C₃ alkyl, R³ is at position h and is C₁-C₆ alkyl; R⁴ is hydrogen; R⁵ is halogen; and n is 2.

20. The method of claim 14, wherein the vascular inflammatory disease is heart disease, stroke, peripheral vascular disease, or vasculitis.
